(19) United States
(12) Patent Application Publication
(10) Pub. No.: US 2024/0226476 A1
(43) Pub. Date: Jul. 11, 2024

(54) SLEEP APNEA PREVENTION AND MONITORING SYSTEM

(71) Applicant: MACKSOUD KHAN, PALO ALTO, CA (US)

(72) Inventor: MACKSOUD KHAN, PALO ALTO, CA (US)

(21) Appl. No.: 18/094,583

(22) Filed: Jan. 9, 2023

(51) Int. Cl. A61M 16/00; A61M 16/08

(52) U.S. Cl. CPC A61M 16/024; A61M 16/0875; A61M 2205/8206; A61M 2230/06; A61M 2230/20; A61M 2230/42; A61M 2230/63

(57) ABSTRACT

The invention provides a sleep apnea prevention and monitoring system for mitigation and treatment thereof. The system comprises a sleep apnea device, a monitoring device operatively connected to the sleep apnea device and preferably a personal mobile device that interacts with the system.

### FIELD OF THE INVENTION

The invention provides a sleep apnea prevention and monitoring system for mitigation and treatment thereof. The system comprises a sleep apnea device, a monitoring device operatively connected to the sleep apnea device and preferably a personal mobile device that interacts with the system.

### BACKGROUND OF THE INVENTION

The tongue and soft palate play a major role in the production of snoring and obstructive sleep apnea, and their participation is needed to causes these symptoms to exist. Hence, it is imperative to have knowledge about these structures, which play a major important role in snoring and obstructive sleep apnea. Most of us do not think of snoring as something to be overly concerned about. However, frequent, loud snoring may be a sign of sleep apnea, a common and potentially serious disorder in which breathing repeatedly stops and starts as you sleep. These breathing pauses during sleep apnea last between 10 to 20 seconds and can occur up to hundreds of times a night.

Obstructive Sleep Apnea (OSA) is a common medical condition that can be quite severe. About one out of twenty-two Americans (about 12,000,000) are reported to have OSA, with many cases undiagnosed. Chronic fatigue has long been recognized as a hallmark of OSA, but a large number of recent clinical studies have shown a large link between OSA, stroke, and death.

Although sleep apnea is treatable, it often goes unrecognized. Untreated sleep apnea can be dangerous and detrimental to your health, so it's important to see a doctor if you suspect that you or a loved one might have it Read on to learn the warning signs of sleep apnea, how to distinguish it from normal snoring, the medical treatments available, and what you can do to help yourself.

Obstructive sleep apnea is a condition in which air flow in the breath is halted or reduced due to narrowing, obstruction, or loosening of the airway. People with OSA symptoms often begin to snore violently soon after sleeping. Snoring will typically be louder and louder. Snoring is then interrupted during long periods of silence without breathing. This is followed by a loud jet and a blow as the person tries to breathe. This pattern repeats. Many people wake up in the morning and feel drowsy throughout the day. This is called Excessive Daytime Sleepiness (EDS). People with sleep apnea may present with irritability or irritability of the spleen, forgetfulness, may sleep or feel sleepy while working, reading or watching television, or even sleep while driving a vehicle, or have headaches that are difficult to treat. OSA patients may also experience depression, which causes worse, hyperactivity behavior (especially in children) or swelling of the legs (if severe).

Many CPAP devices and alternatives to CPAP have been developed, but all have significant disadvantages. Less invasive attempts in the treatment of OSA, such as behavioral changes, sleep positioning, and detachable splints worn in the mouth, have had little effect. A number of different surgical approaches for treating OSA have also been attempted, some of which remain in use. For example, Uvula Palatoplasty (UPPP) and Laser Assisted Uvula Palatoplasty (LAUP) are currently used. However, surgical procedures are often very invasive and not always effective in treating OSA.

What is therefore needed is a system that addresses all of the above issues and seeks to both mitigate and monitor the effects of sleep apnea in users and patients who suffer therefrom.

### SUMMARY OF THE INVENTION

Accordingly, the invention provides a sleep apnea prevention and monitoring system for mitigation and treatment thereof. The system comprises a sleep apnea device having a blower; one or more air filters; a power source; a hose; a mask; a universal connector for the hose and the mask; and electronics usable to control the speed of the blower.

As further constituent of the system herein, a monitoring device is held adjacent to a which analyzes and measures a user's rest and analyzes and measures the use of the sleep apnea device. In practice, the monitoring device is operatively connected to the sleep apnea device.

Ideally, the monitoring device is a ring that is placeable onto one of the user's fingers. The ring records sleep associated biometric data from the user while he or she rests. The ring captures key biometric data including, but not limited to, user movement, a user's sound of snoring, user blood oxygen levels, and the user's heart rate.

The system herein may further incorporate the use of a personal mobile device which is owned by or otherwise under the control of a user or even a medical professional who monitors the user's rest. In practice, the personal mobile device is operationally connected to the sleep apnea device and the monitoring device.

The sleep apnea device of the sleep apnea prevention and monitoring system is preferably battery powered though it may also use a plug-in cord for an electric source. For greatest convenience, the sleep apnea device is mountable to a wall or bed frame very close to a user's head when the user sleeps. Also preferably provided for the sleep apnea device herein a hose which is twist resistant.

### BRIEF DESCRIPTION OF THE FIGURES

The various exemplary embodiments of the present invention, which will become more apparent as the description proceeds, are described in the following detailed description in conjunction with the accompanying drawings, in which:

FIG. 1 is a schematic of the system herein; and

FIG. 2 is an isometric view of the ring device herein.

### DETAILED DESCRIPTION OF THE INVENTION

The following description is provided as an enabling teaching of the present systems, and/or methods in its best, currently known aspect. To this end, those skilled in the relevant art will recognize and appreciate that many changes can be made to the various aspects of the present systems, and/or methods described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

As used throughout, the singular forms “a,” “an” and “the” include plural referents unless the context clearly dictates otherwise. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might,” or “may,” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods.

Thus, if there are a variety of additional steps that can be performed it is understood that each of these additional steps can be performed with any specific aspect or combination of aspects of the disclosed methods.

The invention herein provides a sleep apnea prevention and monitoring system for mitigation and treatment thereof. The system comprises a sleep apnea device having a blower; one or more air filters; a power source; a hose; a mask; a universal connector for the hose and the mask; and electronics usable to control the speed of the blower.

As further constituent of the system herein, a monitoring device is held adjacent to a which analyzes and measures a user's rest and analyzes and measures the use of the sleep apnea device. In practice, the monitoring device is operatively connected to the sleep apnea device.

Ideally, the monitoring device is a ring that is placeable onto one of the user's fingers. The ring records sleep associated biometric data from the user while he or she rests. The ring captures key biometric data including, but not limited to, user movement, a user's sound of snoring, user blood oxygen levels, and the user's heart rate.

The system herein may further incorporate the use of a personal mobile device which is owned by or otherwise under the control of a user or even a medical professional who monitors the user's rest. In practice, the personal mobile device is operationally connected to the sleep apnea device and the monitoring device.

The sleep apnea device of the sleep apnea prevention and monitoring system is preferably battery powered though it may also use a plug-in cord for an electric source. For greatest convenience, the sleep apnea device is mountable to a wall or bed frame very close to a user's head when the user sleeps. Also preferably provided for the sleep apnea device herein a hose which is twist resistant.

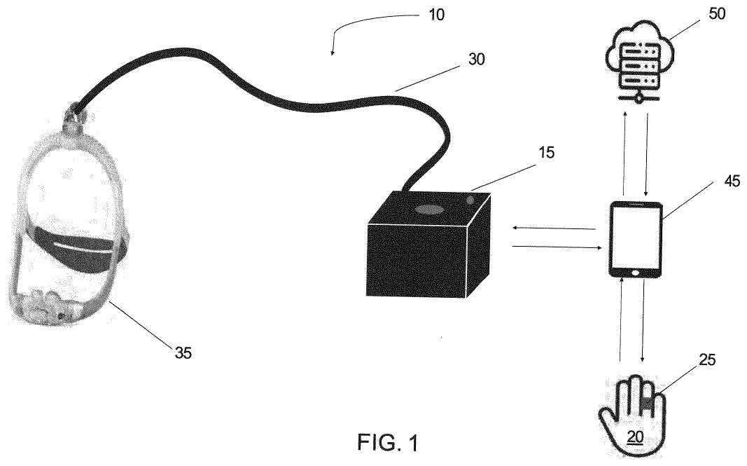

FIG. 1 is a schematic of the system for sleep apnea prevention and monitoring system 10 (or “system 10”) herein. System 10 comprises sleep apnea (and monitoring device) 15 which has a blower for pushing air therethrough, one or more air filters, a power source (either corded or battery), hose 30, mask 35 and electronics within sleep apnea device 15. As shown, sleep apnea device 15 is connected by hose 30 to mask 35.

Also shown in FIG. 1 are user's hand 20 upon which is monitoring device 25, which is preferably a ring placed upon a user's finger. An example of a suitably preferred monitoring device 25 herein is the OURA® ring. Other tech wearable items may be more of the watch type like the APPLE WATCH®, WHOOP® wearable, or FITBIT® wearable, all three of which are worn about a user's wrists.

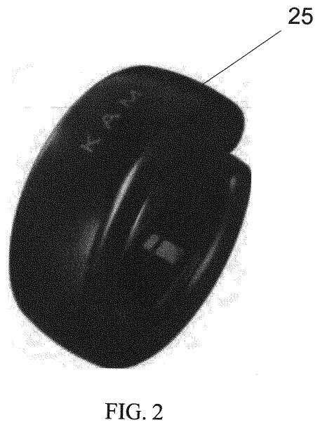

FIG. 2 is an isometric view of monitoring device 25 herein. Monitoring device 25 may comprise a sleep score (1-100) tells a user how well he/she sleeps. The score is based upon how long a user spends in each sleep stage and a user's biometrics like body temperature and heart rate. Using monitoring device 25, a user can tell if a specific activity, like a late nigh coffee use, late night workout, alcohol use, and the like, can affect the quality of one's rest.

Also, monitoring device 25 tracks your sleep quality and sleep stages with research-grade sensors. For example, time of deep sleep, REM sleep, and light sleep every night are recorded and communicated and suggestions for improving each are provided herein. More specifically, monitoring device 25 herein is designed, through use of its hardware and software, to learn about a user's individual sleep patterns and also share personalized guidance on when to start winding down to improve your sleep quality. Importantly, monitoring device 25 herein empowers system 10 can tell when you might be experiencing breathing disturbances which could impact a user's sleep or health.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A sleep apnea prevention and monitoring system, comprising:
a. A sleep apnea device having
i. A blower;
ii. One or more air filters;
iii. A power source;
iv. A hose;
v. A mask;
vi. A universal connector for said hose and said mask;
vii. Electronics usable to control the speed of said blower; and
b. A monitoring device held adjacent to a user, said monitoring device analyzing and measuring the rest of said user, analyzing and measuring the use of said sleep apnea device, said monitoring device being operatively connected to said sleep apnea device.

2. The sleep apnea prevention and monitoring system of claim 1 wherein said monitoring device is a ring placeable onto one of said user's fingers.

3. The sleep apnea prevention and monitoring system of claim 2 wherein said ring records sleep associated biometric data from user while said user rests.

4. The sleep apnea prevention and monitoring system of claim 3 wherein said ring captures key biometric data including, but not limited to, user movement, a user's sound of snoring, user blood oxygen levels, and said user's heart rate.

5. The sleep apnea prevention and monitoring system of claim 1 wherein said system further comprises a personal mobile device.

6. The sleep apnea prevention and monitoring system of claim 5 wherein said personal mobile device is operationally connected to said sleep apnea device and said monitoring device.

7. The sleep apnea prevention and monitoring system of claim 1 wherein said sleep apnea device is battery powered.

8. The sleep apnea prevention and monitoring system of claim 7 wherein said sleep apnea device is wall-mountable.

9. The sleep apnea prevention and monitoring system of claim 1 wherein said hose of said sleep apnea device is twist resistant.
